(19) United States
(12) Patent Application Publication
(10) Pub. No.: US 2018/0015299 A1
(43) Pub. Date: Jan. 18, 2018

(54) ANTI-AGING DEVICE FOR STIMULATING SCALP AND HAIR HEALTH

(71) Applicant: INO BEAUTY INC., Miami, FL (US)

(72) Inventor: Nour Kawa, Miami, FL (US)

(21) Appl. No.: 15/547,672

(22) PCT Filed: Feb. 3, 2016
(86) PCT No.: PCT/US16/16345

§ 371 Date: Jul. 31, 2017

Related U.S. Application Data
(60) Provisional application No. 62/111,499, filed on Feb. 3, 2015.

(51) Int. Cl. A61N 5/06; A61F 7/00; A61H 23/00; A61N 1/36; A61N 1/04; A61N 1/32

(52) U.S. Cl. CPC A61N 5/0617; A61N 1/0484; A61N 1/322; A61H 23/00; A61N 1/326; A61N 1/36014; A61F 7/00; A61F 2007/0087; A61F 2007/0008; A61H 2201/0214; A61H 2201/10; A61H 2205/021; A61N 2005/0644; A61N 2005/0647; A61N 2005/0663; A61N 2005/0652

(57) ABSTRACT

A device for stimulating hair growth, nurturing hair, maintaining existing hair, elongating the hair life cycle and keeping hairs in the active growth stage and/or decreasing the number of hairs in the dying stage, can include one or more radiation sources emitting radiation having a wavelength in a range of about 430 nm to about 495 nm; one or more radiation sources emitting radiation having a wavelength in a range of about 630 nm to about 700 nm; and one or more electrodes extending from the device and configured to contact a scalp of a user to apply a microcurrent to the scalp.

### CROSS REFERENCE TO RELATED APPLICATION

The benefit of priority under 35 U.S.C. §119(e) of U.S. Provisional Patent Application No. 62/111,499 filed Feb. 3, 2015, is hereby claimed and the disclosure is incorporated herein in its entirety.

### BACKGROUND

### Field of the Disclosure

The disclosure relates to a device for achieving one or more of nurturing scalps, stimulating hair follicles, stimulating hair growth, and for strengthening and nurturing existing hair.

### Brief Description of Related Technology

There are many reasons that hair follicles atrophy. For example, follicular atrophy can be the result of a natural condition in which hair gradually thins with age, with more follicles going into a resting phase and remaining hairs becoming shorter and fewer in number. Follicular atrophy can also be the result of a genetic condition that can affect both men and women. There are a number of treatment options available for stimulating hair topical formulations, neutraceuticals and pharmaceuticals, and laser treatments.

Devices for combating hair loss include low-level laser diode therapy. For example, a current conventional device includes a helmet-like device, a comb, or a brush that is worn or used to expose the scalp to low-level light therapy using a combination of red laser and LED light diodes. Such conventional devices typically utilize a single mode of therapy, such as light therapy, and require users to apply the device for 15 to 25 minutes three times a week.

### SUMMARY

There remains a need for an efficient and effective device that can be utilized easily by a user for reasonably durations of time that would ensure better compliance with a treatment regime. It has been advantageously discovered that by combining therapeutic modalities into a single device can results in an efficient and convenient device for the user. In various embodiments, the combination can result in improved results as compared to a single therapy device, such as a light-therapy device. The combination can also, in various embodiments, allow for improved results with reduced duration of use needed, which can aid with user compliance.

In accordance with an embodiment of the disclosure, a device can include one or more radiation sources emitting radiation having a wavelength in a range of about 430 nm to about 495 nm; one or more radiation sources emitting radiation having a wavelength in a range of about 630 nm to about 700 nm; and one or more electrodes extending from the device and configured to contact a scalp of a user to apply a microcurrent to the scalp. In various embodiments, the device can have a power of about 5 watts to about 20 watts.

In accordance with another embodiment, the device can optionally include a cooling element to cool the scalp and/or hair and/or a vibrating element to apply vibration to the scalp and/or hair.

In accordance with an embodiment of the disclosure, a method for stimulating hair can include exposing at least a portion of a user's scalp and hair to a radiation having a wavelength of about 430 nm to about 495 nm; exposing at least a portion of a user's scalp and hair to a radiation having a wavelength of about 630 nm to about 700 nm; and applying at least a portion of the user's scalp and hair to a microcurrent having an intensity in a range of about 300 mA to about 500 mA.

### BRIEF DESCRIPTION OF THE DRAWINGS

FIG. 1A is a top view of a brush in accordance with an embodiment of the disclosure;

FIG. 1B is a side view of the brush of FIG. 1A;

FIG. 1C is a bottom view of the brush of FIG. 1A;

FIG. 2A is a top view of a brush in accordance with another embodiment of the disclosure;

FIG. 2B is a side view of the brush of FIG. 2A;

FIG. 2C is a bottom view of the brush of FIG. 2A;

FIG. 3A is a top view of a brush head in accordance with yet another embodiment of the disclosure; and

FIG. 3B is a side view of the brush head of FIG. 3A.

FIG. 4 is side view of a brush in accordance with an embodiment of the disclosure including a cooling cradle.

### DETAILED DESCRIPTION

Disclosed herein is a device and method that can nurture the scalp, stimulate hair follicles, stimulate hair growth, and strengthen and nurture existing hair. The device and methods disclosed herein can improve hair growth in some embodiments. The device and method can also aid in combating scalp aging, which can improve overall scalp health even in individuals not currently experiencing hair thinning. In various embodiments, the device can result in the appearance of improved hair volume immediately after use, which can be aesthetically pleasing to the user.

As used herein, the term “stimulate hair” or stimulating hair” or “hair stimulation” refers to stimulation of the scalp, hair follicles, and hair. The device can advantageously be used to maintain or improve scalp health, maintain and/or stimulate hair strength and growth, prevent or reduce hair thinning, and/or combat hair loss. It has been advantageously found that the combination of light therapy using wavelengths in both the red and blue spectrums can provide for improved stimulation of hair and scalp, thereby improving overall scalp and hair health. The device can in various embodiments result in improved hair growth. In accordance with various embodiments of the disclosure, the device can also include current stimulation and/or cold therapy in combination with the light therapy to stimulate hair growth and to provide nourishment to the scalp and hair follicle. Without intending to be bound by theory, it is believed that the device provides for improved hair stimulation by one or more of the following: helping to calm the scalp, stimulate the scalp and hair follicle, stimulate cellular regeneration, strength existing hair follicles to generate healthier hair from existing follicles, elongate the natural active hair growth/hair cycle and by stimulating healing. It is also believed that the device can provide for one or more of decreased inflammation, reduced bacterial growth and/or elimination of bacteria, which can aid in reducing, eliminating, and/or preventing dandruff or a dry flaky scalp.

The device can be provided in a variety of forms including, but not limited to, brushes, combs, hair bands, helmets, ties, and hats. The device can be conveniently provided as a cordless device that is battery operated to allow for ease of use. Alternatively, corded device are also contemplated herein.

The device can include one or more radiation sources to provide the light therapy. The radiation sources can emit radiation of the same wavelength, for example, in the red or blue wavelength. Alternatively, the radiation sources can emit radiation of different wavelength. For example, the device can include a portion of the lights sources having a wavelength of about 630 to 700 nm (i.e., red light) and a portion of the radiation sources having a wavelength of about 430 to 495 nm (i.e. blue light). Other suitable wavelengths for the red light spectrum can include about 630 to 660 nm. Other suitable wavelengths for the blue light spectrum can include about 430 to about 485 nm. In an alternative embodiment, the device can include radiation sources of a single wavelength or range of wavelengths corresponding to a given color of the visible light spectrum. For example, the device can include one or more radiation sources each emitting a wavelength in the red light spectrum range or the blue light spectrum range.

Any suitable number and type of radiation sources can be used. For example, the radiation sources can be light emitting diodes (LEDs) and/or heat emitting diodes. For example, the device can include 2 to 20 radiation sources, 3 to 15 radiation sources, 6 to 12 radiation sources, and 1 to 10 radiation sources. Other suitable amount of radiation sources include about 2, 3, 4, 5, 6, 7, 8, 9, 10, 11, 12, 13, 14, 15, 16, 17, 18, 19, and 20. In one embodiment, the device includes 6 LEDs, with three of the LEDs emitting radiation having a wavelength in the red light range and three LEDs emitting light having a wavelength in the blue light range.

In various embodiments, the LEDs can have a power output of about 100 mW to about 700 mW, about 100 mW to about 500 mW, about 300 mW to about 500 mW, or about 250 mW to 400 mW. Other suitable power outputs for the LEDs include about 100, 150, 200, 250, 300, 350, 400, 450, 500, 550, 600, 650, and 700 mW. In various embodiments, the LEDs can output about 15 to 30 lumens, about 20 to 25 lumens, about 15 to 20 lumens, or about 20 to 30 lumens. Other suitable outputs include about 15, 16, 17, 18, 19, 20, 21, 22, 23, 24, 25, 26, 27, 28, 29, and 30 lumens. Without intending to be bound by theory, it is believed that the use of such LEDs can allow for good penetration of the irradiation into the scalp, as well as providing good irradiation coverage to effectively irradiate multiple hair follicles and account for variations in the direction of growth of the hair follicles.

In an embodiment, the device includes 1 to 10 radiation sources emitting light having a wavelength in the red light range and 1 to 10 radiation sources emitting light having a wavelength in the blue light range. Any suitable division of the red light and blue radiation sources can be used. For example, the device can include 4 to 7 red radiation sources and 3 to 5 blue radiation sources. It has been advantageously found that the combination of red light and blue light therapy can provide for improved stimulation for hair growth. Without intending to be bound by theory, it is believed that the radiation sources emitting radiation in the red light spectrum can stimulate the hair follicles, aid in locking a treatment formula, if applied, into the hair strands, and decrease inflammation and redness of the scalp. Without intending to be bound by theory, it is believed that the radiation sources emitting radiation in the blue light spectrum can aid in killing bacteria on the scalp.

In various embodiments, the device can further include radiation sources that emit heat and/or the that emit both light and heat.

The radiation sources can be arranged in a variety of configurations. For example, the radiation sources can be arranged down the center of the device. In such embodiments, for example, a comb or a brush, the radiation sources can be arranged to provide substantially uniform exposure of the portion of the scalp engaged by the device. In other embodiments, for example, in embodiments in which the device engages the entire scalp, the light devices can be uniformly dispersed across the device to provide uniform exposure to the entire scalp. Alternatively, a device can include the radiation sources disposed in a desired region and the device can be adjustable such that affect areas of the scalp can be engaged by the device and exposed to the light form the radiation sources.

The one or more radiation sources can include one or more lenses to focus the irradiation from the light source onto the hair follicles. For example, a domed lens can be used in various embodiments.

The device can operate using any suitable optical power level. For example, the device can operate at an optical power level of about 60 to 80 mW/cm3.

The device can further include one or more bristles and/or nodes. The nodes can function similar to bristles. Any suitable number of bristles and/or nodes can be included on the device depending on the configuration of the device. It is also contemplated herein that the device can be free of bristles and/or nodes. The bristles and/or nodes, if present, can provide a massaging attribute, which can aid in stimulating hair growth. In various embodiments, an electrode can be incorporated into the bristle and/or node to provide a current stimulation to the scalp. Alternatively, the current stimulation can be provided by an electrode or other structure, separate from the bristles and/or nodes. For example, in various embodiments the device can include 0 to 30 bristles and/or nodes, 5 to 25 bristles and/or nodes, 10 to 20 bristles and/or nodes, or 0 to 22 bristles and/or nodes. All or a portion of the bristles and/or nodes can include an electrode. Alternatively, none of the bristles and/or nodes can include an electrode.

The device can include one or more electrodes for providing electrical stimulation to the scalp in combination with the light therapy. As indicated above, the electrodes can be provided as separate structures on the device or can be incorporated into one or more bristles or teeth of a comb. The electrodes, whether provided as a separate structure or incorporated into a bristle or comb tooth can extend from the device so as to contact the scalp when the device is applied to the scalp. The device can include any suitable number of electrodes. For example, the device can include 1 to 10 electrodes, 2 to 8 electrodes, 3 to 6 electrodes, 6 to 10 electrodes, or 3 to 7 electrodes. Other suitable numbers of electrodes include 1, 2, 3, 4, 5, 6, 7, 8, 9, and 10 electrodes. The electrodes can provide a current, such as but not limited to microcurrent, galvanic current, or sonic pulsing current, to the scalp. The current can have an intensity of about 100 to 800 μA, about 100 to 500 μA, about 300 to 500 μA, about 300 to 600 μA, about 300 to 700 μA, about 300 to 800 μA, about 300 to 600 μA, about 100 to 300 μA, and about 200 to 500 μA. Other suitable intensities include about 100, 200, 300, 400, 500, 600, and 700 μA. For example, in an embodiment, the device can include microcurrent having an intensity in a range of about 300 μA to about 700 μA. For example, in an embodiment the device can include a microcurrent having an intensity of 400 μA LA. For example, in another embodiment, the device can include a galvanic current having an intensity in a range of about 300 μA to about 800 μA. For example, in an embodiment, the device can include a pulsing current having an intensity of about 300 μA to about 600 μA. In various embodiments, the device can be used in combination with a hair care product that can conduct energy from the applied micro-current or other current into the scalp. The inclusion of a current can advantageously provide the device with a therapeutic benefit that can aid in relaxing the user's scalp during use of the product, which in turn can give the user an overall sense of relaxation.

In various embodiments, the device can further include a heat energy transfer module. The heat energy transfer module can direct energy to the heat the upper dermis of the scalp, well below the surface of the scalp. It is believe that such heating can stimulate collagen renewal, which can improve the scalp health and combat scalp aging.

In various embodiments, the device can further include a cooling element. The cooling element can be configured to operate during light stimulation and microcurrent stimulation. Alternatively, the cooling operation can be configured to operate after the light stimulation and/or the microcurrent stimulation. Operation of the cooling element after light and and/or microcurrent stimulation can beneficial result in restriction of the blood vessels beneath the skin of the scalp. Without intending to be bound by theory, it is believed that this restriction of the blood vessels may contribute to the improved effectiveness of the stimulation therapy achieved by the device in accordance with embodiments of the disclosure.

Any suitable cooling mechanisms as known in the art can be included. For example, the device can include a cooling cartridge that when triggered emits a temporary burst of cooling gas onto the scalp. The cartridge can be configured to emit the burst of cooling gas or a flow of cooling gas. The flow of gas can be directed to the scalp for any suitable duration, for example, about 1 min to about 5 min, about 1 min to about 3 min, or about 2 min to about 4 min. Other suitable times include, about 1, 2, 3, 4, and 5 minutes. The cooling gas can be generated by a canister or cartridge structure that resides within the device or is external to the device. The canister or cartridge includes a pressurized cooled gas that can be triggered for release through outlets in the device head that direct the cooled gas to the scalp. The cartridge or canister for cooling can be mounted in the device, for example, using a Peltier junction. The canister or cartridge can be disposed in or on the device such that it is replaceable or rechargeable. In an embodiment, the canister is a dimethyl ether canister. The canister can be fluidly coupled to outlets using a manifold or any other known structure.

In yet another embodiment, the device can include a snap on or otherwise removable cooling head adapter that can be placed in a cooling apparatus, such as a freezer, to be cooled when not in used and then placed on the device in the cooled state for use.

In alternative embodiment or as an additional feature, the electrodes can be cooled while the device is not in use and impart cooling to the scalp during application of the LED and/or microcurrent stimulation. Cooling from the electrodes can also be applied while the LED and/or microcurrent stimulation is turned off, such that cooling from the cooled electrodes is the only therapy being applied for a given duration.

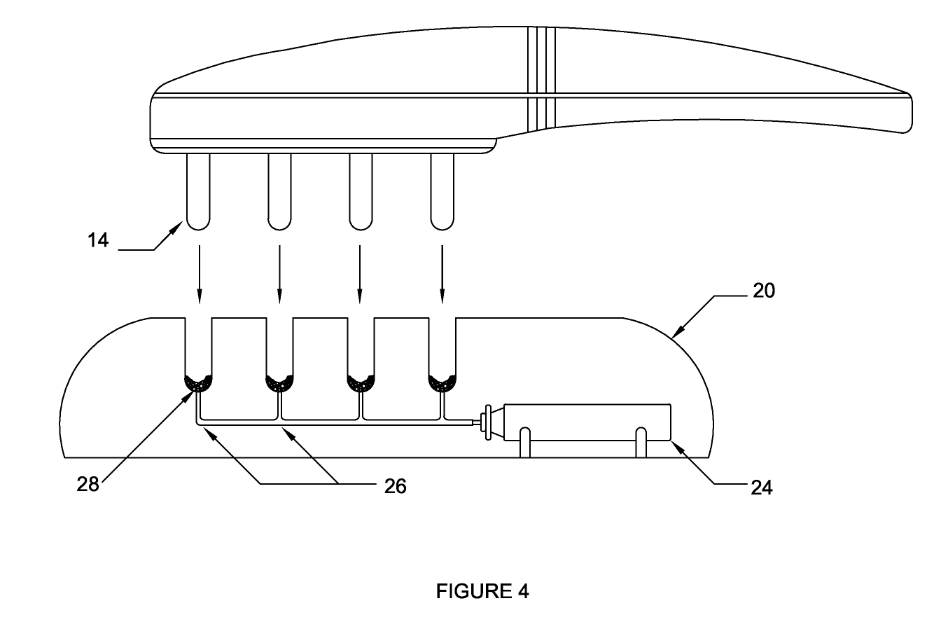

For example, referring to FIG. 4, the device can be provided with a cradle 20 that receives the device 10, such that the electrodes 14 reside in cooling wells 22 of the cradle 20 when resting on the cradle 20. In other embodiments, the brush can include cooling extensions, such as metal nodes, separate from the electrodes, which can be received in the wells for cooling as opposed to or in addition to the electrodes. The cooling wells 22 can be fluidly coupled to a cooling canister 24 that emits cooled gas to the wells when the electrodes are residing in the wells. The cooling canister and the cooling wells 22 can be fluidly coupled, for example, by a manifold 26 or any other know structure for fluidly coupling two regions. The wells 22 can include an insulating material 28 within the wells to help retain the cooled imparted from the cooling canister. For example, the insulating material can be sponges disposed in the region in which the cooled gas enters the wells, as shown in FIG. 4. Any suitable cooling gasses and canisters can be used as is known in the art. For example, the wells can be fluidly coupled to a dimethyl ether canister by a manifold 26.

The cooling element can aid in cooling the device as well as provide a cooling therapy to the scalp. Without intending to be bound by theory, it is believed that a cooling therapy may aid in closing the hair follicles post stimulation to aid in retaining nutrients added to the hair follicles, for example, from a treatment formulation, after the stimulation. The device can be cooled to any suitable temperature, for example about 32° F. to about 60° F., or about 40° F. to about 60° F. Other suitable temperatures include, about 32, 34, 36, 38, 40, 42, 44, 46, 48, 50, 52, 54, 56, 58, and 60° F.

In various embodiments, the device can be configured to vibrate. In embodiments in which the device includes bristles and/or comb teeth, one or more of the bristles and/or comb teeth can include a vibration element that allows for vibration of the one or more bristles and/or comb teeth. In other embodiments in which the device does not include bristles and/or comb teeth, the device itself can be configured to vibrate. In various embodiments, the device can include sonic vibration, for example, in a range of 0 to 16 kilohertz.

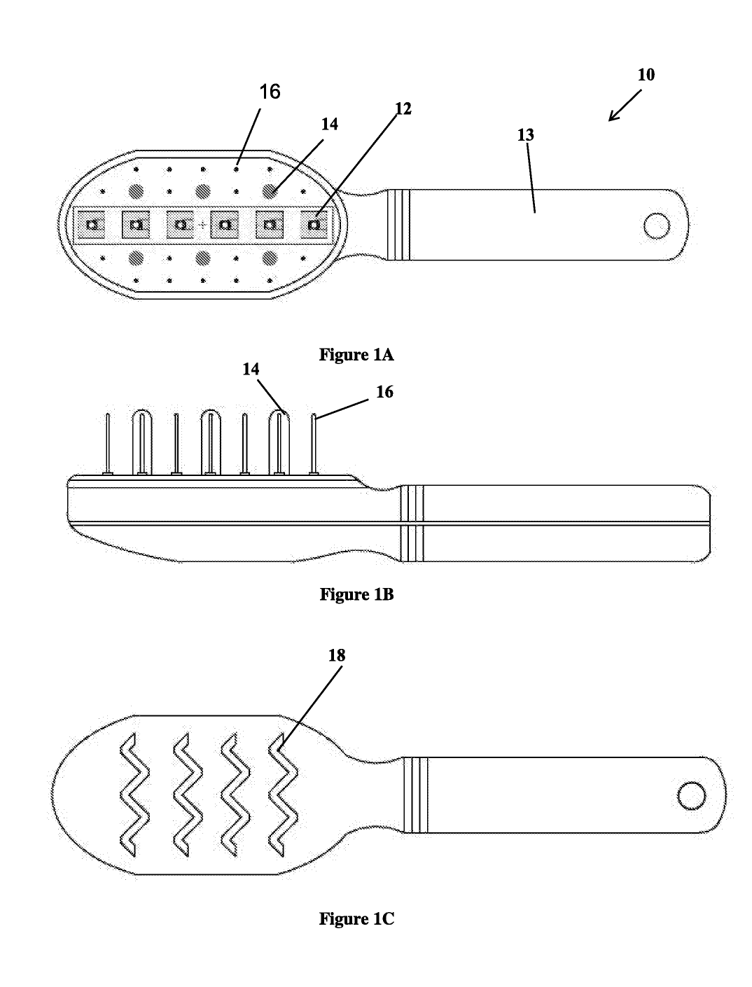

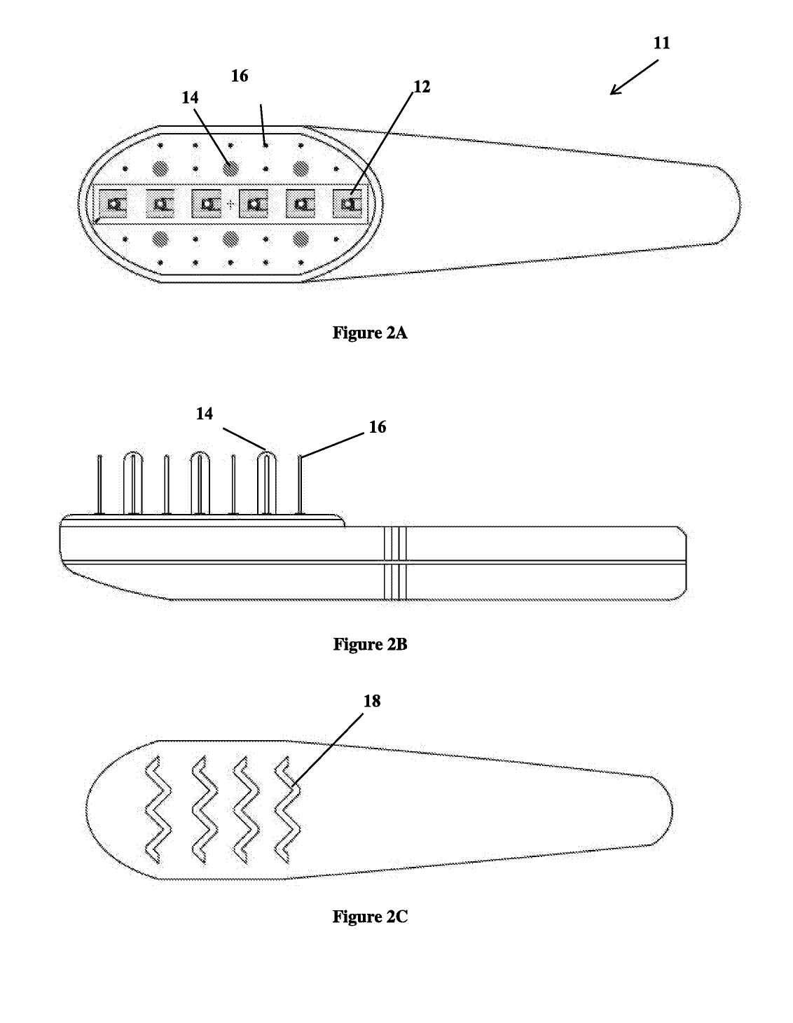

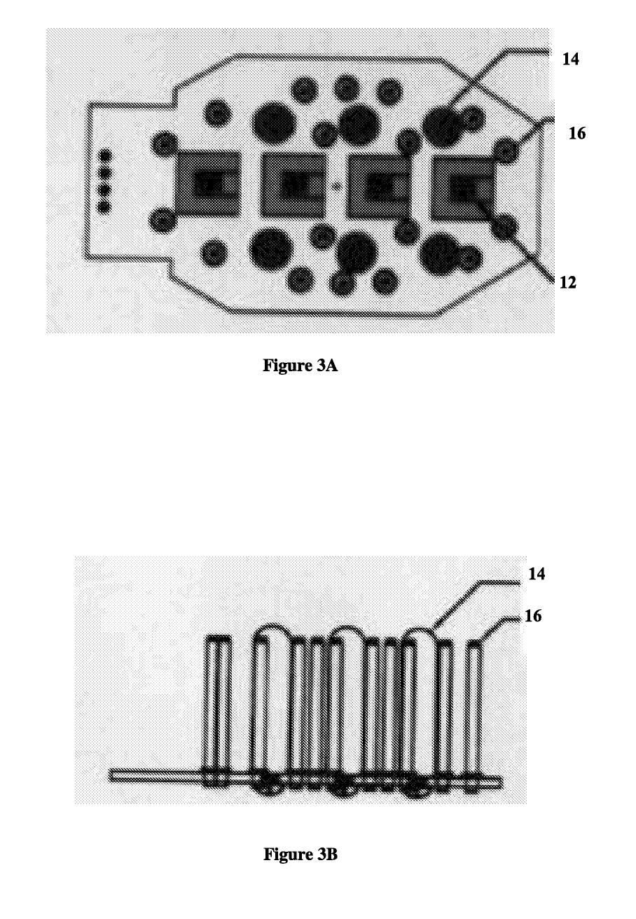

The device can optionally include any further structures, such as power devices, control circuits, and heat sinks necessary for operation of electrodes and LEDs, as is known in the art. For example, as shown in FIGS. 1 and 2, the device can include one or more elements to aid in dissipation of heat from the radiation sources and/or electrodes. These heat dissipating elements can be provided on any suitable surface of the device and can be electrically connected to the radiation sources and/or electrodes.

Other elements such as timers, automatic operation control systems, and/or programmable control systems can be included. For example, the device can be configured to operate such that the scalp is exposed to the light and microcurrent therapy substantially simultaneously. Alternatively, the device can be programmed to expose the scalp to the light therapy for a duration of time and then expose the scalp to the microcurrent therapy for a duration of time, or vice versa. If other therapies, such as cooling or vibration therapies are included in the device, such timers or programmable controls can be utilized to allow for operation of such elements in combination with one or both of the light therapy and the microcurrent therapy or before or after such therapies.

The device can further include a user interface to allow for selection of the therapies activated on the device. For example, the device can include radiation sources, a electrodes, a vibrating element and/or a cooling element, and the user interface can allow the user to select for use any one or more of the therapies. The user interface can also allow the user to configure the duration of the one or more therapeutic elements of the device and allow for tailoring of which therapeutic elements are utilized in combination or in serial operation.

Referring to FIGS. 1A-1C, an embodiment of a brush device 10 in accordance with the disclosure is shown. The device of FIG. 1 includes LEDs 12 emitting light in the red and/or blue wavelengths and a plurality of electrodes 14 that are sized to contact the scalp and provide microcurrent stimulation to the scalp. The device may also include a plurality of bristles 22 to allow the device to be used as a brush and provide added benefits of a massage therapy by the brushing action. As shown in FIG. 1C, the device includes one or more heat dissipation elements 18 that aid in dissipating heat from the electrical structures of the device. While a brush type device with bristles is shown, the device can also be provided in the form of a comb and other non-bristled structures.

FIGS. 2A-2C illustrate a brush device 11 in accordance with another embodiment of the disclosure. The device 10 can be sized and shaped to be conveniently held by the user for easy operation. The device 10 can have any suitable handle 13 for gripping, and can contain the radiation sources and microcurrents within the brush head. The radiation sources and electrodes can be arranged on the brush head to provide a window treatment area that can substantially uniformly expose the engaged portion of the scalp and hair to the light and microcurrent therapies. For example, in an embodiment a brush device can include a treatment area of about 10 cm² to about 15 cm², about 11 cm² to about 12 cm². In an embodiment, the treatment area of the brush device is about 11.5 cm².

In a comb device, for example, the radiation sources 12 can be positioned between teeth of the comb. One or more electrodes 14 can be incorporated into the teeth or alternatively placed adjacent to the teeth.

It has been advantageously discovered that by combining the radiation sources with the microcurrent, effective stimulation of the hair and scalp can be achieved while reducing the overall number of radiation sources needed. Conventional devices utilize 40 more or more radiation sources to provide stimulation. Such a vast number of radiation sources requires a large power output, making it difficult if not impossible to provide a compact device that has multiple therapeutic modes. It was conventionally thought that such a large number of light stimulation sources was needed to effective stimulate the scalp. The device in accordance with the disclosure is capable of generating effective stimulation with a reduced number of radiation sources, which reduces the power needs, allowing for incorporation of other therapeutic modes, such as microcurrent and/or cooling, which can be run simultaneously on the device. In various embodiments, the device is a hand-held device such as brush or comb.

A method for stimulating hair and/or improving scalp health in accordance with an embodiment of the disclosure can include exposing at least a portion of a user's hair and scalp to radiation having a wavelength in a range of about 430 nm to about 495 nm (blue light), exposing at least the portion of the user's hair and scalp to radiation having a wavelength in a range of about 630 nm to about 700 nm (red light), and applying a microcurrent to the user's scalp. In accordance with an embodiment of the disclosure, the user's scalp can be exposed to the blue light radiation and the red light radiation at substantially the same time and for substantially the same duration. In accordance with another embodiment, the user's scalp can be exposed to the blue light radiation and the red light radiation for at different times and/or different durations. The method can alternatively include applying only a single radiation, for example radiation in the red light range or radiation in the blue light range. In accordance with an embodiment of the disclosure, the microcurrent can be applied to the scalp at substantially the same time as the light exposure or at a different time as the light exposure. For example, in an embodiment, the method can include substantially simultaneously exposing at least a portion of the user's scalp to red light and blue and applying a microcurrent. Any suitable order or combination of the light and microcurrent therapies can be used.

The method can further include applying one or more additional therapies to the scalp, including, but not limited to massage therapy, vibration therapy, and cooling therapy. The massage therapy can be provided by a brushing or combing action using a device having bristles or comb teeth. The vibration therapy can be provided by vibration of the device, either in its entirety or a portion of the device, such as, for example, one or more bristles or comb teeth. The cooling therapy can be provided by a cooling element on the device and can be applied during or after the other therapies. In various embodiments, the method include utilizing a first set of therapies for a first time and applying the cooling therapy after, for example, substantially immediately after, the first set of therapies. The device can be cooled across all or a portion of the treatment area.

In accordance with an embodiment of the disclosure, the method can include exposing the scalp and hair to a first set of therapies for a first duration. The first set of therapies can include radiation-based stimulation and microcurrent stimulation, as described above. In another embodiment, the first set of therapies can include radiation-based stimulation. Any of the other stimulations or therapies described above can be combined with the first set of therapies or can be applied as a second set of therapies. The first duration can be for example, about 5 mins to about 20 mins, about 7 mins, to about 15 mins, about 8 mins to about 11 mins, about 5 mins to about 15 mins, or about 7 mins to about 12 mins. Other suitable times include, about 5, 6, 7, 8, 9, 10, 11, 12, 13, 14, 15, 16, 17, 18, 19, and 20 mins. In various embodiments, the first duration can be less than 20 mins, less than 15 mins, or less than 12 mins.

The method can further include a second therapy or set of therapies that is applied for a second duration. The second therapy can be, for example, cooling therapy in which the scalp is cooled for the second duration. The second duration can be when cooling is applied, for example, about 3 min. Other times are contemplated herein depending on the therapy included for the second therapy and are described herein with reference to the specific therapies.

In various embodiments, the device can include a control system that automatically stops the first set of therapies after the first duration. In various embodiments, the control system can engage the second set of therapies after completion of the first set of therapies and automatically stop the second therapy or set of therapies after the second duration.

Any number of therapies or sets of therapies can be utilized for any predetermined durations. For example, the method can include engaging a first set of therapies, a second set of therapies, and a third set of therapies. Other embodiments can include multiple cycles of the same therapies. For example, the method can include stimulating with light and microcurrent, cooling, re-stimulating with light and microcurrent, and re-cooling.

Any combination of therapies or order of therapies can be used. For example, a method utilizing a cooling therapy can include such cooling therapy after application of light therapy. Alternatively, in various embodiments, the device can operate all therapies at substantially the same time and/or duration.

Application of the therapy can depend on the configuration of the device. For example, in a brush type device, the user can be instructed to brush a first side of their head for a preset time and then brush a second side of the head. The device can include timing devices to indicate when a switch should be made from one side to the other. Additional aids to the user including a rate sensor for sensing the rate of brushing and signaling to the user when a rate is too fast or too slow can also be included in the device. The signaling can be in the form of lights or sounds to engage the user's attention.

The device can also include the ability for the user to select the therapies or sets of therapies to be applied and the durations in which the therapy should be engaged. This can provide a user with a customizable device capable of tailoring the therapy and duration to their needs.

### EXAMPLES

Devices in accordance with embodiments of the disclosure were tested and compared to a commercially available laser-therapy comb device. 100 men and 100 women, age 18-75 years old, were divided into 5 test groups, with 20 subjects in each group and balanced for mean age and gender. All subjects were healthy and had complaint of progressive hair thinning or loss for more than 2 years and were diagnosed clinically by their dermatologist as having androgenic alopecia.

Group I was a control group. Group II utilized a device that provided red LED as the stimulation therapy. Group III utilized a device that provided red and blue LED as a light stimulation therapy combined with a microcurrent stimulation. Group IV utilized a device that provided blue LED as the stimulation therapy. Group V utilized the commercial laser-therapy comb. All items were placed in a cold container before use and cooled to a temperature of 40-50° F. before use.

The devices in accordance with the disclosure were utilized for 12 minutes, five times a week and the commercial device was used for 15 minutes, five times a week. The devices were used under the direct supervision of clinical laboratory personal to ensure compliance.

The devices in accordance with the disclosure included 9 LEDS with 22 bristles. Blue LEDs has a wavelength in a range of 430 nm to 485 nm and red LEDs had a wavelength of 630 nm to 660 nm. The device emitting a mixture of red and blue radiation included 3 LEDS having a wavelength of 630 nm to 660 nm (red) and 3LEDS having wavelength of 430 nm to 485 nm (blue). The device emitting microcurrent included 6-10 electrodes for imparting a microcurrent of 300 mA to 500 mA to the scalp when in use. The device was configured to apply light stimulation and current stimulation at substantially the same time.

After 90 days of use, users were asked for their subjected evaluation of the performance of the device in accordance with the disclosure.

| Group No. | Subjective Evaluation Results |
|---|---|
| Group II | 85% Women and 85% Men believed that they |
| | saw noticeable results |
| | 85% Women and 85% Men believed that their |
| | hair looked longer |
| | 85% Women and 85% Men believed that their |
| | hair looked fuller |
| | 100% Women and 85% Men believed that |
| | their hair looked shinier |
| | 85% Women and 75% Men believed that their |
| | hair broke less |
| | 95% Women and 100% Men said that the |
| | device was easy to use |
| | 85% Women and 85% Men were overall |
| | pleased with the results |
| Group III | 95% Women and 85% Men believed that they |
| | saw noticeable results |
| | 95% Women and 90% Men believed that their |
| | hair looked longer |
| | 95% Women and 90% Men believed that their |
| | hair looked fuller |
| | 100% Women and 100% Men believed that |
| | their hair looked shinier |
| | 100% Women and 90% Men believed that |
| | their hair broke less |
| | 100% Women and 100% Men said that the |
| | device was easy to use |
| | 95% Women and 85% Men were overall |
| | pleased with the results |
| Group IV | 85% Women and 85% Men believed that they |
| | saw noticeable results |
| | 85% Women and 85% Men believed that their |
| | hair looked longer |
| | 85% Women and 85% Men believed that their |
| | hair looked fuller |
| | 100% Women and 85% Men believed that |
| | their hair looked shinier |
| | 85% Women and 75% Men believed that their |
| | hair broke less |
| | 95% Women and 100% Men said that the |
| | device was easy to use |
| | 85% Women and 85% Men were overall |
| | pleased with the results |
| Group V | 85% Women and 85% Men believed that they |
| | saw noticeable results |
| | 90% Women and 90% Men believed that their |
| | hair looked longer |
| | 90% Women and 85% Men believed that their |
| | hair looked fuller |
| | 100% Women and 85% Men believed that |
| | their hair looked shinier |
| | 85% Women and 80% Men believed that their |
| | hair broke less |
| | 95% Women and 100% Men said that the |
| | device was easy to use |
| | 85% Women and 85% Men were overall |
| | pleased with the results |

After 90 days of use, the device utilized in Group III (current plus mixed LEDs) was found to perform statistically better than the light alone brushes. Additionally testing was completed with a device of Group III which was cooled in a cooling apparatus and cooled to 40-50° F. prior to use. An overall improvement of approximately 35% in new hair growth, split end reduction, follicle thickness, growth rate was found compared to the radiation only devices and the commercial device.

While various embodiments have been described above, the disclosure is not intended to be limited thereto. Variations can be made to the disclosed embodiments that are still within the scope of the appended aspect.

1. A device, comprising:
one or more radiation sources emitting radiation having a wavelength in a range of about 430 nm to about 495 nm; and/or
one or more radiation sources emitting radiation having a wavelength in a range of about 630 nm to about 700 nm; and
one or more electrodes extending from the device and configured to contact a scalp of a user to apply a current to the scalp.

2. The device of claim 1, further comprising a cooling element.

3. A device, comprising one or more radiation sources emitting one or more radiation sources emitting radiation having a wavelength in a range of about 430 nm to about 495 nm;
one or more radiation sources emitting radiation having a wavelength in a range of about 630 nm to about 700 nm;
one or more electrodes extending from the device and configured to contact a scalp of a user to apply a current to the scalp; and
a cooling element configured to cool the user's scalp.

4. The device of claim 2, wherein the cooling element is configured to expose the user's scalp to cooling gas having a temperature of 32° F. to 60° F.

5. The device of claim 2, wherein the cooling element is provided as a cradle having wells that receives the electrodes to cool the electrodes by a cooling gas or fluid emitted into the wells.

6. The device of claim 1, further comprising a vibrating element.

7. The device of claim 1, further comprising one or more bristles.

8. The device of claim 6, wherein the one or more bristles comprise the electrode for applying the microcurrent to the scalp.

9. The device of claim 1, wherein the device is in the form of a brush or the device is in the form of a comb or the device is in the form of a hat.

10. (canceled)

11. (canceled)

12. The device of claim 1, wherein the device comprises 6 radiation sources emitting radiation having a wavelength in a range of about 430 nm to about 495 nm and 4 radiation sources emitting radiation having a wavelength in a range of about 630 nm to about 700 nm.

13. The device of claim 1, further comprising a heat transfer module configured to heat the upper dermis of the scalp when the device is used.

14. The device of claim 1, wherein the electrodes apply a current having an intensity in a range of about 300 μA to about 600 μA.

15. A method for stimulating hair, comprising:
exposing at least a portion of a user's scalp and hair to a first radiation having a wavelength of about 430 nm to about 495 nm;
exposing the at least a portion of a user's scalp and hair to a second radiation having a wavelength of about 630 nm to about 700 nm;
applying to the at least a portion of the user's scalp and hair to a current having an intensity in a range of about 300 μA to about 500 μA.

16. The method of claim 15, further comprising cooling the at least a portion of the user's scalp and hair after at least one of exposing the at least a portion of the user's scalp to a radiation having a wavelength of about 430 nm to about 495 nm, exposing the at least a portion of the user's scalp to a radiation having a wavelength of about 630 nm to about 700 nm, and applying the microcurrent.

17. The method of claim 15, further comprising cooling the at least a portion of the user's scalp and hair substantially simultaneously with at least one of exposing the at least a portion of the user's scalp to a radiation having a wavelength of about 430 nm to about 495 nm, exposing the at least a portion of the user's scalp to a radiation having a wavelength of about 630 nm to about 700 nm, and applying the microcurrent.

18. The method of claim 16, comprising cooling the user's scalp for about 1 min to about 3 min.

19. The method of claim 15, comprising exposing the user's scalp to the first and second radiations and applying the current substantially simultaneously.

20. The method of claim 15, comprising exposing the user's scalp to the first and second radiations and applying the current for a duration of 5 mins to 20 mins.

21. The method of claim 20, comprising exposing the user's scalp to the first and second radiations and applying the current for a duration of 8 mins to 11 mins.

22. The method of claim 15, further comprising applying vibration to the at least a portion of the user's scalp and
